(19) United States
(12) Patent Application Publication
(10) Pub. No.: US 2020/0387335 A1
(43) Pub. Date: Dec. 10, 2020

(54) DISPLAY SYSTEM AND PROCESS FOR DISPLAYING AN OUTPUT OF AN ELECTROMEDICAL DEVICE

(71) Applicant: Drägerwerk AG & Co. KGaA, Lubeck (DE)

(72) Inventors: Stefan SCHLICHTING, Lubeck (DE); Sina SCHEUPLEIN, Lubeck (DE)

(21) Appl. No.: 16/891,724

(22) Filed: Jun. 3, 2020

(30) Foreign Application Priority Data
Jun. 7, 2019 (DE) 102019003996.3

(51) Int. Cl. G06F 3/14; G16H 15/00; G06F 21/31

(52) U.S. Cl. CPC G06F 3/14; G06F 21/31; G16H 15/00

(57) ABSTRACT

A display system (100) displays an electromedical device (105) output. A display unit (110) receives first and second display signals (112), (114) and displays a combined visual output (116). A display signal unit (130) communicates with the electromedical device and receives device data (107) via a communication interface (132), assigns the device data to a visualization type and determines a respective display type, dynamically, as a function of the visualization types to be displayed currently, and generates and outputs the first display signal based thereon. A data processing unit (140) reads out data set data (142) and generates and outputs the second display signal from the data set data. A logon screen (144) is outputted in case an authorization state of the display system assigned to these data types is not currently present for currently read-out data set data from a predefined subset of data types.

### CROSS REFERENCE TO RELATED APPLICATIONS

This application claims the benefit of priority under 35 U.S.C. § 119 of German Application 10 2019 003 996.3, filed Jun. 7, 2019, the entire contents of which are incorporated herein by reference.

### TECHNICAL FIELD

The pertains to a display system for displaying an output of at least one electromedical device and to a corresponding process for displaying an output of at least one electromedical device. Furthermore, the present invention pertains to a program with a program code for carrying out the process according to the present invention.

### TECHNICAL BACKGROUND

Using a display unit that displays the data of a connected electromedical device and is synchronized with the mode of operation of this electromedical device is known in the medical sector.

U.S. Pat. No. 9,104,789 B2 teaches that a display unit receives data from different connected electromedical devices and displays them in a variable manner. In this connection, alarm information is displayed in a separate, expanded window of a control menu of the display unit.

US 2016/0128647 A1 describes a medical display system that receives received data from different connected electromedical devices and outputs them sorted according to their importance.

### SUMMARY

An object of the present invention is to provide an improved output of at least one electromedical device, and especially an output of data of at least one electromedical device, which output is compatible with other devices.

A display system for displaying an output of at least one electromedical device, with a display unit, with an input unit, with a display signal unit and with a data processing unit, is provided according to the present invention for accomplishing this object.

The display unit is configured to receive a first display signal and at least one second display signal and to display a combined visual output based on the two display signals.

The input unit has a user interface, via which a user can provide a user input.

The display signal unit has a communication interface for communication with the at least one electromedical device and is configured to receive device data that pertain to the electromedical device via the communication interface, to assign these device data to a visualization type and to determine a respective display type of the received device data dynamically as a function of the visualization types to be displayed currently. Furthermore, the display signal unit is configured to generate the first display signal based on the device data and on the display type determined in each case and to output same to the display unit.

The data processing unit is configured to read out data set data and to generate and to output the second display signal from these data set data. In this case, the data processing unit is configured to generate the second display signal such that a logon screen to be filled out by a user of the display system via the input unit is outputted in case an authorization state of the display system assigned to these data types is not currently present for currently read-out data set data from a predefined subset of data types.

It was discovered within the framework of the present invention that when a separate display system is used with respect to the electromedical device, it must be ensured that received data are outputted visually in a suitable manner. In this connection, it was further discovered that work is done with personalized data in routine clinical practice, for which a suitable data protection should be ensured. For this purpose, the provision of a combined visual output is provided, in which both device data of an electromedical device, for example, physiological measured values and data set data, which indicate a logon screen to be filled out, for example, in case of a lack of authorization, are displayed together.

Furthermore, it is advantageously ensured that the display system according to the present invention is operated independently of electromedical devices.

It is especially advantageous for the display system according to the present invention that received device data, i.e., for example, physiological measured values, are displayed, while read-out data set data are blocked by a logon screen in case the authorization state that is necessary for these data is not currently present. As a result of this, a fast medical procedure by medical staff is made possible, even if this medical staff is not authorized to access the data set data. Furthermore, fast treatment is made possible in case of an emergency, without a change in the authorization state having to be made via the logon screen beforehand, even if the treating person would be authorized to access the data set data.

For example, the data set data, which are processed by the data processing unit from the second display signal, are administrative data of the patient treatment, for example, demographic data of the patient.

Another advantage of the display system according to the present invention is the classification of the received device data into different visualization types, so that a respective display type can be determined dynamically based on the visualization types to be displayed currently. The display type typically comprises a display position of the device data to be outputted, a display size of these device data, a display font of these device data and/or a display color of the device data.

The device data comprise all data, which are received by the at least one electromedical device and are differentiated from the data set data within the framework of the present invention by the latter being read out and processed by the data processing unit. The data set data may be received, for example, by at least one external system, which is not an electromedical device. As an alternative or in addition, the data set data may originate from a data set that is provided by a memory module of the data processing unit. The at least one external system is typically a patient management system, which outputs person-specific data as data set data.

The reading out of the data set data may comprise all types of a provision of the data set data for the data processing unit. In particular, the reception of a corresponding signal and the reading out of the data received by such a signal are reading out in the sense of the present invention.

The provision of a logon screen to be filled out advantageously makes it possible for only users who are authorized to see defined person-specific information to be able to see this information by means of the display system. The display system according to the present invention makes possible an especially high level of data protection as a result.

A simultaneous outputting of device data of a plurality of different electromedical devices is made possible by an advantageous compatibility of the display system according to the present invention with different electromedical devices. Furthermore, a complicated coordination between the display unit and a respective electromedical device is avoided due to the display system according to the present invention.

The individual units of the display system according to the present invention may each be arranged at a spaced location from one another or within a common housing. In this case, the processing of the device data may advantageously be carried out by a single processor. In addition, it may be advantageous that the processing of other data set data is also carried out on the same processor.

The display system according to the present invention may be arranged at a spaced location from the at least one electromedical device.

The dynamic determination of the respective display type comprises a determination of the display type as a function of the assigned visualization types corresponding to a currently present number of visualization types. Thus, the dynamic determination takes place again after the reception of data and after a corresponding assignment of a visualization type in each case. Consequently, no fixed display location is typically present for a defined visualization type, since this visualization type is likewise dynamically fixed within the framework of the dynamically fixed display type.

All display properties as a whole, for example, display position, display shape, display style and/or display size, form the respective display type of the device data received.

The device data received are, for example, physiological parameters, which are determined by the electromedical device, and/or a setting of the electromedical device, for example, a current mode of operation, an alarm limit, a configuration and/or a current patient profile, and/or an alarm that was determined by the electromedical device.

According to the present invention the second display signal and thus the display of the data set data cannot be influenced by the display signal unit.

Preferred embodiments of the display system according to the present invention will be described below.

At least one of the two display signals preferably indicates a sequence of individual images, a video data stream and/or a website data stream.

The first display signal and/or the second display signal are preferably generated at predefined time intervals, and especially periodically. The predefined time intervals are selected here such that they correspond to a typical duration of clinically relevant changes of the device data generated by the electromedical device. The predefined time intervals are advantageously at least 0.1 sec.

The visual output is preferably carried out via a display of the display unit. The display is a touch display in an especially preferred variant. In an especially preferred variant of this embodiment, the touch display additionally forms the user interface of the input unit via which the user input is carried out.

The display is preferably enclosed by a housing of the display unit.

The user input may be embodied, for example, by the input of a user identification, the reading out of a key card and/or the reading out of biometric data of a user. Corresponding embodiments of user interfaces are generally known and are therefore not explained in detail below.

In an especially preferred embodiment, the display system further has a display signal monitoring unit, which is connected for signal technology to the display unit and to the display signal unit and which is configured to receive the first display signal from the display signal unit and to receive an output signal from the display unit. In this case, the output signal contains information about the combined visual output carried out. The display signal monitoring unit is further configured to compare the first display signal and the output signal with one another based on the respectively determined display type and to output a warning signal in case of a lack of agreement. It can be especially advantageously ensured in this embodiment that the output of the device data and the output of the data set data are carried out such that an overlap of data is avoided and/or especially relevant data are not overlapped by other data. As a result, the data set data of the data processing unit and the device data of the electromedical devices can be displayed within the framework of the combined visual output in a manner coordinated especially well with one another. This coordinated combined visual output is especially advantageously carried out by a change in the defined display type by the display signal unit being triggered by the outputted warning signal. The avoidance of overlaps of data is made possible in this embodiment due to adaptation of the output of the device data. In an alternative and/or additional variant, the warning signal is outputted directly to a user of the display system. Such an output to the user can be embodied, for example, by a display of an error message via the display unit.

A lack of agreement is determined by the display signal monitoring unit in case the combined visual output carried out drops below a predefined level of agreement with the device data of the first display signal. Thus, a lack of agreement is present, for example, if device data from a predefined subset of especially relevant data types are not displayed in the combined visual output visible to the user. Consequently, it is advantageously ensured that especially relevant device data are not overlapped by data set data, especially administrative data, for example, a logon screen, a user ID, patient information and/or a device ID. The predefined level of agreement may pertain, in addition to the contents of the device data, also to a visual similarity, for example, in case of a comparison of the similarity of font type to be displayed and displayed font type, or to the detectability of display device data, for example, due to an assessment of a present lack of contrast between a display color and a background color.

A lack of agreement between the combined visual output carried out and the output of the device data to be carried out is present in an embodiment if relevant device data, for example, clinical data, are not displayed or are only partially displayed within the framework of the visual output due to an overlapped display of other data. In another embodiment, it is ensured by this comparison of visual output carried out and visual output to be carried out that the displayed device data and especially displayed symbols can be distinguished from other device data, for example, a different number, a different letters or a different symbol.

The comparison for detecting lack of agreement is preferably carried out by checking of individual partial areas of the combined visual output and assignment of these partial areas to data indicated by the first display signal. As an alternative or in addition, the display of individual data from the first display signal is checked by a corresponding checking of the combined visual output indicated by the output signal. In another embodiment, the checking of individual partial areas is implemented by a pixel by pixel checking of the displayed combined visual output.

The display signal monitoring unit is further configured to check whether or not the output signal has data set data or device data of a visualization type, which may be outputted exclusively in the presence of a defined authorization state and to output a corresponding warning signal in case this authorization is not currently present in a variant of the above-described embodiments. As a result of this, it is ensured that a user cannot see protected data without corresponding authorization. This variant advantageously combines the advantage of the display signal monitoring unit that the data of the first display signal and the second display signal are combined such that a coordinated combined visual output is possible due to a change in the display type of the device data with the advantage of searching for protected data in the combined visual output. The display signal monitoring unit is for this purpose preferably configured to determine data set data or device data displayed by the output signal and to assign same to a visualization type. As an alternative or in addition, the display signal monitoring unit is configured to read out the visualization type assigned to the corresponding data displayed in the combined visual output based on the output signal.

In another advantageous embodiment, the data processing unit is further configured to receive an authorization signal depending on the user input and to determine a currently present authorization state of the display system as a function of the authorization signal, an output of the corresponding data set data being carried out for a predefined subset of data types via the second display signal only if an authorization state assigned to these data types is currently present. In this embodiment, an output is carried out for a predefined subset of data types to the output unit by the second display signal only in the presence of a corresponding authorization state. The current authorization state is advantageously fixed by the user input. There are at least two different authorization states, namely a basic state, which indicates that the user is not authorized to see confidential data, and a first state, which indicates that the user is authorized to see a predefined subset of data types which contain confidential data. A distinction is preferably made between other states, so that only a small group of people is authorized to see all data set data. Such a gradation of different authorization states advantageously makes it possible to control which persons have access to certain confidential data, for example, personal data. As a result of this, the display system according to the present invention makes possible an especially high level of protection of personal data, especially while maintaining the display of the device data.

In another preferred embodiment, the predefined subset of data types comprises personal data of a person being treated with the electromedical device. The handling of such data may be subject to legal regulations, so that a verification of what group of people had access to what confidential data may be advantageous for complying with such legal regulations. Moreover, the display system according to this embodiment makes it possible to output treatment-relevant device data, while the person-specific data of the person being treated, which are not relevant to the acute treatment of the person, are not outputted as the combined visual output. In another exemplary embodiment, the predefined subset of data types comprises personal data of physicians who used the electromedical device for treatment in the past. The predefined subset of data types comprises a treatment history of the person being treated with the electromedical device in another additional or alternative embodiment.

In another embodiment of the display system according to the present invention, the display signal unit is further configured to generate the first display signal such that a corresponding reference on the combined visual output is provided in case not all device data received from the electromedical device can be visually outputted by the display unit because of the dimensions of the combined visual output. As a result of this, a user of the display system according to the present invention is informed that not all received data are displayed via the display unit. In a preferred variant of this embodiment, device data not displayed can be displayed by an interaction between the user and the display unit. Such an interaction is, for example, the touching of a corresponding control surface on a touch display of the display unit. In another example of this variant, such an interaction is the use of a scroll bar.

The display signal unit is further configured to determine a display size and/or a display position of received device data of a visualization type as a function of a relevance level assigned to this visualization type in another preferred embodiment. In this embodiment, especially relevant pieces of information are advantageously displayed differently than less relevant information. In particular, especially relevant information can be displayed by a larger font size and/or by an especially central position within the combined visual output. A number of displayed data can be increased by using different display sizes. In particular, less relevant information can be displayed by a smaller font size than relevant information. Consequently, a large number of less relevant information can be displayed at the same time without the display of relevant information being compromised thereby. In an alternative and/or additional embodiment, the display type comprises a display style as display property in addition to the display size and the display position. The display style indicates, for example, a font type to be used and/or a font color to be used for the information to be displayed. Information is defined here and hereinafter as information indicated by received data in each case.

In an especially preferred embodiment of the display system according to the present invention, the communication interface is configured to receive device data from a plurality of electromedical devices. In this case, the visualization type assigned to the device data by the display signal unit preferably comprises device information, which indicates the electromedical device corresponding to the device data. The fact that the display system according to the present invention functions separately from the electromedical device, the device data of which shall be provided by the display system, is especially advantageously exploited in this embodiment. The visualization type assigned to these device data comprises a relevance level, which is dependent on the electromedical device corresponding to these device data, in a variant of this embodiment.

The display signal unit preferably receives all device data via the communication interface from electromedical devices within an electromedical communication network. Such an electromedical communication network is assigned to the respective display system and typically comprises a plurality of electromedical devices. In a first example, the electromedical communication network comprises all electromedical devices that are used for the treatment of a single, concrete patient. In a second example, the electromedical communication network comprises all electromedical devices of a single device type that are used on a hospital ward.

The communication interface of the display signal unit according to the present invention preferably makes possible a wireless transmission of data. In an alternative and/or additional embodiment, the communication interface makes possible a cable-based transmission of data. The communication interface makes possible both a wireless transmission of data and a cable-based transmission of data in another embodiment.

In another embodiment, the display unit is configured as arranged at a spaced location from the display signal unit and/or from the display signal monitoring unit and/or from the data processing unit and/or from the input unit. The display signal unit and the display signal monitoring unit are arranged in a common housing and are connected for signal technology to the display unit in a variant of this embodiment. In another embodiment, all units of the display system according to the present invention are arranged in a common housing.

In another embodiment, the display system comprises at least one additional data processing unit, which is configured to output an additional second display signal. The display unit is preferably configured here to receive at least three resulting display signals and to display the combined visual output based on these display signals.

According to another aspect of the present invention, the above-mentioned object is accomplished by a process for displaying an output of at least one electromedical device.

The process according to the present invention has the following steps:
provision of a user interface, via which a user can provide a user input;
reception of device data that pertain to the electromedical device;
assignment of the received device data to a respective visualization type and dynamic determination of a respective display type of the received device data based on the visualization types to be displayed currently;
generation and outputting of a first display signal based on the received device data and on the display type determined in each case;
reading out of data set data;
generation and outputting of a second display signal based on the data set data, wherein the second display signal indicates a display of a logon screen to be filled out in case an authorization state assigned to these data types is not currently present for currently read-out data set data from a predefined subset of data types; and
reception of the first display signal and of the second display signal and displaying of a combined visual output based on the two display signals.

The process according to the other aspect of the present invention especially advantageously makes it possible to use a separate device for the display of device data of an electromedical device. The display of device data of different electromedical devices by a single display medium is, moreover, advantageously made possible. As a result of this, an especially great clarity of the medical device data can be ensured. Furthermore, the process according to the present invention ensures that, in addition to the received device data, data set data, especially non-medical data set data can also be displayed by the display unit.

Finally, the process according to the present invention advantageously allows measured values from electromedical devices to be displayed, while person-specific data that are read out at the same time, especially those received before the reading out, are not outputted and are blocked by the logon screen to be filled out. As a result, a medical care by a person not authorized for data access can be rapidly made possible in case of an emergency, or a rapid medical procedure by a person authorized for data access can be made possible without this person having to provide a user input.

Preferred embodiments of the process according to the present invention are described below.

In an especially preferred embodiment, the process according to the present invention comprises, furthermore, the steps:
sending of an output signal, which contains information about the combined visual output carried out;
reception of the output signal and of the first display signal and comparison of the two signals with one another; and
outputting of a warning signal in case there is a lack of agreement between the combined visual output carried out and the visual output to be carried out according to the first display signal.

This embodiment advantageously makes it possible to continuously check the device data in the combined visual output, so that an error in this output can be detected rapidly. As a result of this, the process according to the present invention according to this embodiment is especially less prone to error in case of the combined visual output. An overlap of device data of the first display signal and of data set data of the second display signal can especially be avoided and/or rapidly corrected.

In another embodiment, the process according to the present invention has the steps:
checking whether or not the output signal has device data or data set data of a visualization type, which may be outputted exclusively in the presence of a defined authorization state; and
outputting of a corresponding warning signal in case this authorization state is not currently present.

It is advantageously ensured in this embodiment that only a group of people authorized for this has access to protected data.

In an advantageous embodiment, the process according to the present invention has, moreover, the steps:
reception of an authorization signal as a function of the user input; and
determination of a currently present authorization state based on the authorization signal, wherein the corresponding data set data are outputted via the second display signal for a predefined subset of data types only if an authorization state assigned to these data types is currently present.

In this embodiment, which data set data may be outputted in view of the authorization state that is currently present is advantageously already taken into consideration during the generation of the second display signal.

In another preferred embodiment, the process is configured to display an output of a plurality of electromedical devices with the additional step
reception of device data that pertain to at least one additional electromedical device, wherein the visualization type assigned to the device data comprises device information which indicates the electromedical device corresponding to the data.

The fact that the process according to the present invention can be carried out independently of the respective electromedical device is advantageously exploited in this embodiment. As a result of this, it is especially possible that device data from the plurality of electromedical devices can be displayed simultaneously by the process according to the present invention. A relevance level or a group of relevance levels of the received data can preferably be inferred due to the respective device information.

Furthermore, the above-mentioned object is accomplished by a program with a program code for carrying out the process according to at least one of the above-mentioned embodiments of the process according to the present invention when the program code is run on a computer, on a processor or on a programmable hardware component.

The program may also execute only a part of the data processing according to the present invention in this case. At least the function of the display signal unit, of the input unit and of the data processing unit are preferably controlled by a program and/or coordinated part of a program. The program according to the present invention is preferably executed by a processor of the display system. As an alternative, the program is executed at least by a first processor of the display signal unit and by a second processor of the data processing unit.

The present invention shall now be explained in more detail on the basis of advantageous exemplary embodiments, which are schematically shown in the figures.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a first exemplary embodiment of a display system according to the present invention;

FIG. 2 is a schematic view of a second exemplary embodiment of the display system according to the present invention;

FIG. 3 is a schematic view of a third exemplary embodiment of the display system according to the present invention;

FIG. 4 is a schematic view of a first exemplary embodiment of a combined visual output of the display system according to the present invention;

FIG. 5 is a schematic view of the combined visual output of the display system according to the present invention for the display of an incomplete output of received device data;

FIG. 6 is a schematic view of the combined visual output of the display system according to the present invention for the display for a Shifted window with data set data; and

FIG. 7 is a flow chart of a first exemplary embodiment of a process according to the present invention.

### DESCRIPTION OF PREFERRED EMBODIMENTS

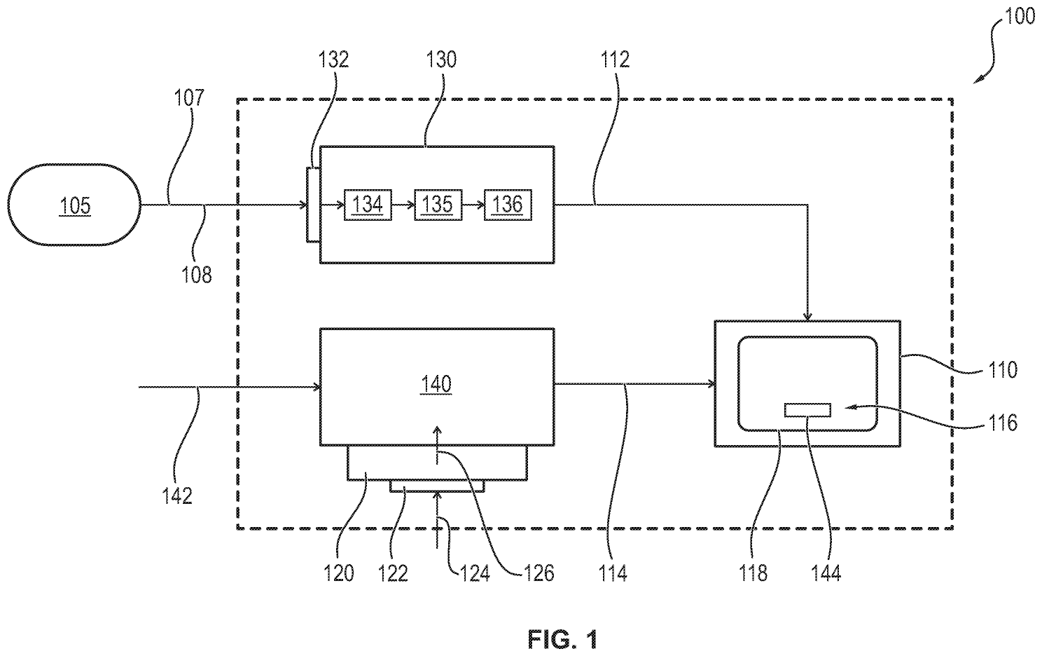

Referring to the drawings, FIG. 1 shows a schematic view of a first exemplary embodiment of a display system 100 according to the present invention.

The display system 100 according to the present invention for displaying an output of at least one electromedical device 105 comprises a display unit 110, an input unit 120, a display signal unit 130 and a data processing unit 140.

The display unit 110 is configured to receive a first display signal 112 and a second display signal 114 and to display a combined visual output 116 based on the two display signals 112, 114. The combined visual output 116 is carried out on a display 118 of the display unit 110.

The input unit 120 has a user interface 122, via which a user of the display system 100 can provide a user input 124. In the present case, the user input is a keyed input of an identification number provided via the user interface 122. In an exemplary embodiment, not shown, the user input is an input due to the reading out of biometric data. In another exemplary embodiment, not shown, the user input is carried out via the reading out of a corresponding identification number of a personal key card. In the present case, the user interface 122 is a keyboard. In an exemplary embodiment, not shown, the user interface is a touch operating element, a computer mouse or a joystick.

The display signal unit 130 has a communication interface 132 for communication with the at least one electromedical device 105. The display signal unit 130 is configured here to receive device data 107 that pertain to the at least one electromedical device 105 via the communication interface 132. For this, the communication interface 132 receives the device signal 108 indicating the device data 107. In the present case, the device signal 108 with the device data 107 is received in a cable-based manner via an Ethernet connection. In an exemplary embodiment, not shown, the device signal is received via a wireless connection, especially via WLAN, Bluetooth, BLE or ZigBee. In another exemplary embodiment, not shown, the cable-based connection is formed by a bus system, for example, by a USB system.

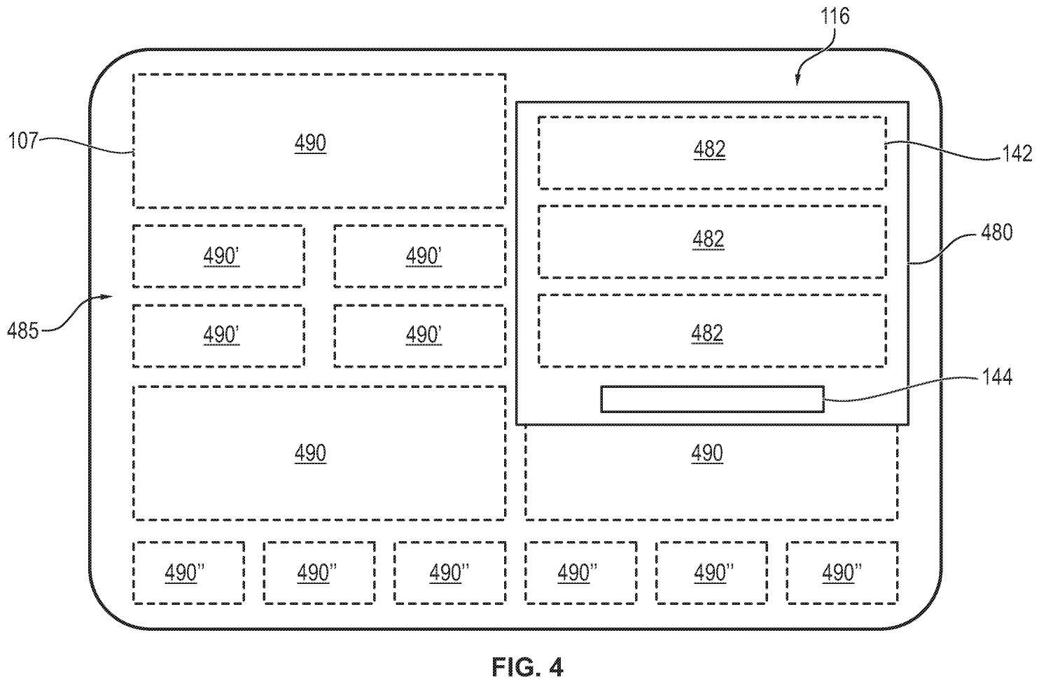

The display signal unit 130 is configured to assign the device data 107 to a visualization type within the framework of a first processing step 134. In a next, second processing step 135, a respective display type of the received device data 107 is dynamically determined as a function of the visualization types to be displayed currently. Based on the device data 107 and the respectively determined display type, the first display signal 112 is generated and outputted to the display unit 110 in a next, third processing step 136 of the display signal unit 130. In the present embodiment, the determined display type of the device data 107 comprises a display position of the device data 107 as a display property. The display position describes a position of the displaying of the corresponding device data 107 within the framework of the combined visual output of the display unit 110. Examples of the display position and of the display property are described within the framework of FIGS. 4 through 6.

The data processing unit 140 is configured to receive data set data 142 and to generate and to output the second display signal 114 from these data set data 142. The data set data 142 are, for example, personal data from a patient management system. In particular, the data set data 142 may further be data which are outputted by an external computer (not shown), for example, by a computer used for management purposes.

The data processing unit 140 is configured here to generate the second display signal 114 such that a logon screen 144 to be filled out via the input unit 120 by a user of the display system 100 is outputted in case an authorization state of the display system 100 assigned to these data types is not currently present for currently received and in this case read-out data set data 142 from a predefined subset of data types. The logon screen 144 forms an input field, in which the user input 124 can be carried out, for example, for inputting a predefined person-specific identification number. After the inputting of a corresponding identification number, the data processing unit 140 would output all data set data 142 that were currently received, for which the user has an authorization corresponding to his identification number. It is ensured as a result that a user cannot access data, for which he has no authorization, through the display system 100 according to the present invention.

In the exemplary embodiment shown, the predefined subset of data types comprises data of a person being treated with the at least one electromedical device 105.

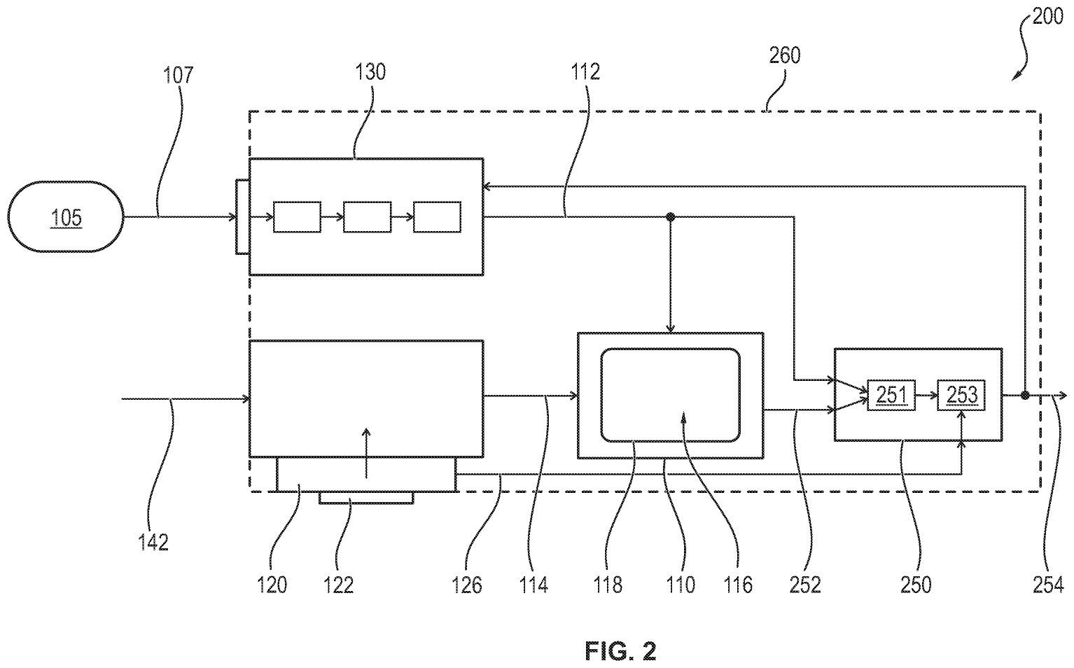

The currently present authorization state is determined in the exemplary embodiment shown by the data processing unit 140. Here, an authorization signal 126 as a function of the user input 124 is outputted by the input unit 120 and received by the data processing unit 140. The currently present authorization state of the display system 100 is then determined as a function of this authorization signal 126. The corresponding data set data 142 are outputted via the second display signal 114 for the predefined subset of data types only if an authorization state assigned to these data types is currently present. In an exemplary embodiment, not shown, all data set data received are comprised in the second display signal and are first removed from the combined visual output 116 by means of the display unit 110 or the display signal monitoring unit explained within the framework of FIG. 2.

The authorization state is determined in the present embodiment by a comparison of the identification number provided via the user interface with a group of identification numbers stored in a memory (not shown), to each of which a corresponding authorization state is assigned.

In an exemplary embodiment, not shown, the authorization state is determined by the input unit and a correspondingly adapted authorization signal, which indicates the currently present authorization state, is outputted.

The received device data 107 are preferably physiological parameters, which were determined by the at least one electromedical device 105 and/or a setting of the electromedical device 105 and/or an alarm that was determined by the electromedical device 105. The electromedical devices 105 may be, for example, patient monitors, anesthesia systems, ventilation systems, thermotherapy devices or infusion pumps. The determined physiological parameters may be, for example, measured values, for example, the invasive or non-invasive blood pressure, heart rate, pulse rate, oxygen saturation, respiration rate, body temperature and/or episodes of arrhythmia. Settings of the electromedical device 105 may be, for example, an alarm limit, a mode of operation, a detected alarm state and/or a state of signaling.

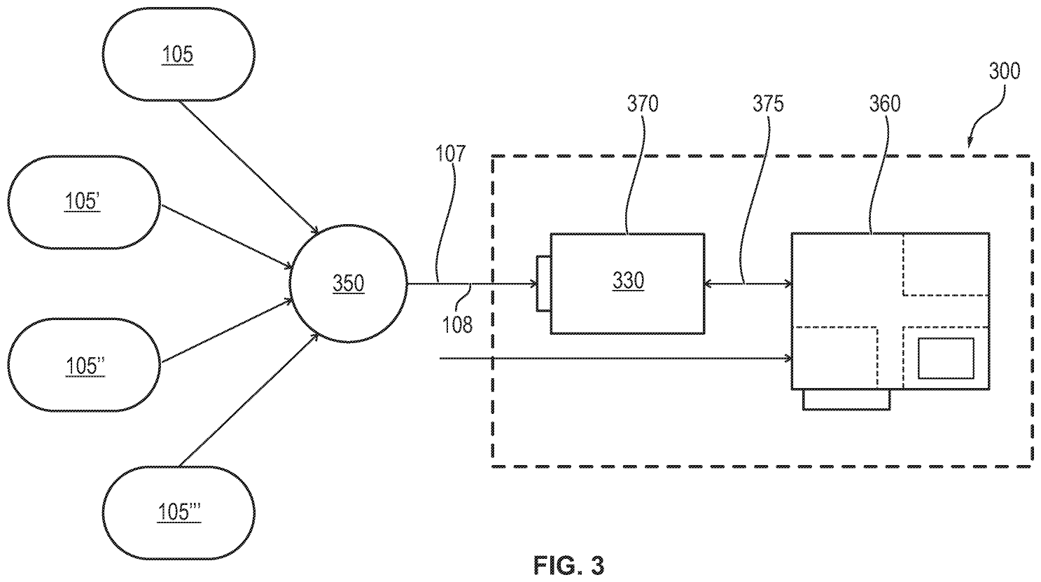

In the exemplary embodiment shown, all units 110, 120, 130 and 140 of the display system 100 are arranged in a common housing. In another exemplary embodiments, these units are arranged in separate housings, as shown within the framework of FIG. 3.

FIG. 2 shows a schematic view of a second exemplary embodiment of the display system 200 according to the present invention.

The display system 200 differs from the display system 100 shown in FIG. 1 by additionally having a display signal monitoring unit 250.

The display signal monitoring unit 250 is connected for signal technology to the display unit 100 and to the display signal unit 130. In the exemplary embodiment shown, it is a cable-based connection. Furthermore, the display signal monitoring unit 250 is configured to receive the first display signal 112 from the display signal unit 130 and to receive an output signal 252 from the display unit 110. The received first display signal 112 may differ from the first display signal 112 outputted to the display unit 110, especially differ in regard to signal duration and output frequency, wherein the two first display signals 112 both have, however, as common signal components information regarding the display types of the received device data. In this connection, the output signal 252 indicates information about the display position and other display properties of all data 107, 142 outputted within the framework of the combined visual output 116 carried out. The display signal monitoring unit 250 is further configured to compare the received first display signal 112 and the output signal 252 with one another in a comparison step 251 based on the respectively determined display type and to output a warning signal 254 in case of a lack of agreement.

The warning signal 254 is outputted to a user of the display system 200 as an optical signal (not shown) in the present case. In addition or as an alternative, the warning signal 254 is outputted to the display signal unit 130 in this exemplary embodiment; as a result, the display signal unit 130 can carry out a correction of the first display signal 112 in an automated manner.

The comparison of the combined visual output 116 carried out and a visual output to be carried out also comprises according to the present invention a checking by the display signal monitoring unit 250 whether or not all letters, numbers and/or symbols to be displayed are visible to the user. Thus, it is seen whether a font type used for the display of the data is currently unsuitable, whether data are completely or partially overlapped due to an output of other data and/or whether a font color is currently unsuitable for the display of the data. Due to the combination of the second display signal 114 and the first display signal 112, an overlapping in case of the display of data and in this case especially an at least partial overlapping of device data 107 by data set data 142 are possible. Thus, the display signal monitoring unit 250 advantageously makes it possible to ensure that no especially relevant device data 107 are overlapped by the data set data 142 of the second display signal 114.

In principle, the output of the data set data 142 remains uninfluenced by the display signal monitoring unit 250 according to the present invention. Only the output of the device data 107 can be influenced by the warning signal 254 such that, for example, the display position, the display size and/or another display property of the device data 107 is influenced such that these device data 107 are clearly visible to a user of the display system 200. Such a change in the display property is shown in detail within the framework of FIGS. 4 through 6.

The display signal monitoring unit 250 in the exemplary embodiment shown is further configured to check in a checking step 253 whether or not the output signal 252 has data set data 142 or device data 107 of a visualization type, which may be outputted exclusively in the presence of a defined authorization state. For this, the display signal monitoring unit 250 is configured to determine the visualization type of the outputted data set data 142 based on the output signal 252. The display signal monitoring unit 250 is further configured to receive the authorization signal 126 outputted by the input unit 120 and to determine therefrom the currently present authorization state. As a result, the display signal monitoring unit 250 according to the present invention is configured to detect whether or not currently outputted data set data are to be assigned to a visualization type, which may be outputted exclusively in the presence of a defined authorization state. The display signal monitoring unit 250 is further configured to output a corresponding warning signal 254 in case this authorization state is not currently present.

Furthermore, a housing 260 of the display unit 110, which encloses both the display 118 for the combined visual output 116 and the user interface 122, is shown in FIG. 2.

FIG. 3 shows a schematic view of a third exemplary embodiment of the display system 300 according to the present invention.

The display system 300 differs from the display systems 100, 200 shown in FIGS. 1 and 2 by being configured to display the output of four different electromedical devices 105, 105′, 105″, 105′″. The four different electromedical devices 105, 105′, 105”, 105′ are configured to output their data to a communication network 350. This takes place in the present case by means of a respective wireless connection, for example, via WLAN, Bluetooth, BLE or ZigBee. In the exemplary embodiment being shown, the communication network 350 comprises all the electromedical devices that are used for the treatment of a single patient. In an alternative exemplary embodiment, not shown, the communication network comprises all the electromedical devices that are used for the measurement of a defined physiological parameter or set of parameters on a hospital ward in order to thereby obtain an especially good overview of the corresponding physiological state of the patients of this ward.

The display signal unit 330 is configured here to receive the device signal 108, which comprises the data 107, directly from the communication network 350.

Furthermore, the display system 300 differs from the display systems 100, 200 shown in FIGS. 1 and 2 by the display signal unit 330 being arranged in a separate housing 370, which is arranged at a spaced location from a common housing 360 of the other units of the display system 300. In the exemplary embodiment shown, a bidirectional wireless connection 375 between the display signal unit 330 and a central processor (not shown) within the common housing 360 makes possible a communication according to the present invention between the units. In an exemplary embodiment, not shown, a cable-based bidirectional connection makes possible a communication between the units. In another exemplary embodiment, not shown, the display signal unit communicates with the display unit via a unibidirectional connection.

FIG. 4 shows a schematic view of a first exemplary embodiment of a combined visual output 116 of the display system 200 according to the present invention.

The combined visual output 116 combines in this case an administrative output 480 of the data set data 142 and the logon screen 144 corresponding to the second display signal 114 with the medical output 485 of the device data 107 corresponding to the first display signal. The medical output 485 comprises in the present case the entire part of the combined visual output 116 that does not belong to the administrative output 480.

The combined visual output 116 has a plurality of output areas 490, 490′, 490″ which are shown in dotted lines. The concrete data-specific contents of the output areas 490, 490′, 490″ were not shown for the sake of clarity. The display of dotted or closed borders, which are used below, is only used for better visualization of the output areas 490, 490′, 490″, wherein no borders within the combined visual output 116 are preferably visible to a user of the display system 200. The output areas differ in their size in this case. In the exemplary embodiment shown, the display unit has received three device data, which were assigned to a visualization type, to which a high relevance level is assigned. These three outputs correspond to the three large output areas 490. An example of such a visualization type with a high relevance level is a physiological measured value, for example, the oxygen saturation in the blood. Furthermore, device data with a medium relevance level were received, to which four output areas 490′ of medium size were assigned. Finally, device data with a low relevance level, to which six output areas 490″ of small size were assigned by the display signal unit, were also contained.

The large output areas 490 are arranged both in the upper area and in the central area of the combined visual output 116 and are thereby also rapidly visible to a user of the display system 200 because of their position.

The arrangement of the output areas and selection of the size thereof were dynamically determined by the display signal unit because of the visualization types to be displayed currently. In case the other visualization types were to be displayed currently, the same display system would hence provide a different arrangement of output areas.

The display size and the display position of received data form display properties in this exemplary embodiment. All display properties as a whole form the respective display type of received data. The fact that a font type and a font color of the data to be displayed likewise form a display property is not shown in FIG. 4.

In an exemplary embodiment according to the present invention, not shown, only the display position and information whether a display of the data is carried out form a respective display property of the display type dynamically determined in each case.

The administrative output 480 comprises three administrative output areas 482 of the same size and the logon screen 144. The data set data 142 are displayed in the administrative output areas 482. The display signal unit and the display signal monitoring unit have in the exemplary embodiment shown no influence on the administrative output 480. In an exemplary embodiment, not shown, the display signal monitoring unit is configured to trigger an error output overlapping the administrative output via the display signal unit in case the data set data shown may not be outputted in view of the currently present authorization state.

The display signal monitoring unit has recognized in the exemplary embodiment shown of the combined visual output 116 detected that, in spite of the administrative output 480 and a slight overlapping with an output area 490 with high assigned relevance level, all device data 107 are visible to the user of the display system and hence no change in the first display signal and also no output of a warning signal are necessary. The case of a change in the display position by the display signal monitoring unit because of an overlap is described in FIG. 6.

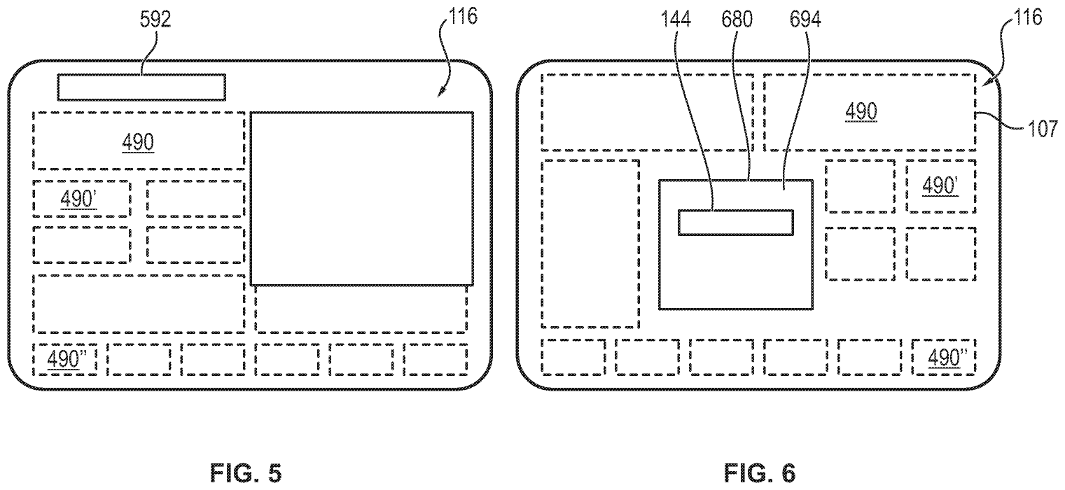

FIGS. 5 and 6 show a schematic view of the combined visual output 116 of the display system according to the present invention for the display of an incomplete output 592 of received data (FIG. 5) and for a shifted window 694 with data set data, which are not device data (FIG. 6).

The combined visual outputs 116 shown in FIGS. 5 and 6 are based on the combined visual output 116 shown in FIG. 4 and show how this combined visual output 116 is changed due to the display of an incomplete output 592 and due to a change in the administrative output 680.

The display of an incomplete output 592 is typically carried out in an edge area of the visual output 116, at the upper edge in the present case. The display of an incomplete output 592 informs a user of the display system that not all the device data received from the display system can be visually outputted by the display unit because of the dimensions of the visual output. This display likewise leads to the fact that the other output areas 490, 490′, 490″ are shifted and made smaller. In the present case, the output areas 490, 490′, 490″ are made smaller such that the size ratios of the output area 490, 490′, 490″ and of the administrative output 480 remained the same among one another.

FIG. 6 shows a combined visual output 116 which is changed compared to FIG. 4 to the extent that the administrative output 680 by the second display signal takes a different display position than in FIG. 4. In the exemplary embodiment of the display system 100 shown in FIG. 1, such a shifting of the administrative output 680 leads to an overlap of this output with the corresponding output areas 490, 490′, 490″ for the device data 107. A defined area of the combined visual output 116 for the administrative output 680 is therefore preferably provided for the display system 100, so that an overlapping takes place only in case of an unforeseen shifting of the administrative output 680. For the exemplary embodiment of the display system 200 shown, the checking of the output signal by the display signal monitoring unit makes possible a correction of the display position of the large and medium output areas 490, 490′ such that an overlapping is avoided. In this case, the display size of these output areas 490, 490′ is also changed such that there is no overlap between the output area and the administrative output 680.

In the exemplary embodiment being shown in FIG. 6, the data processing unit detects that an authorization state of the display system 200 assigned to these data types is not currently present for currently received and read-out data set data from a predefined subset of data types, so that a logon screen 144 to be filled out is outputted instead of these data.

The output areas 490, 490′, 490″ shown are not defined as limiting. In exemplary embodiments, not shown, different sizes, positions and shapes of output areas are provided. The data can in this case be outputted within the output areas in the form of letters, numbers, symbols, tables, diagrams and of any other graphic display.

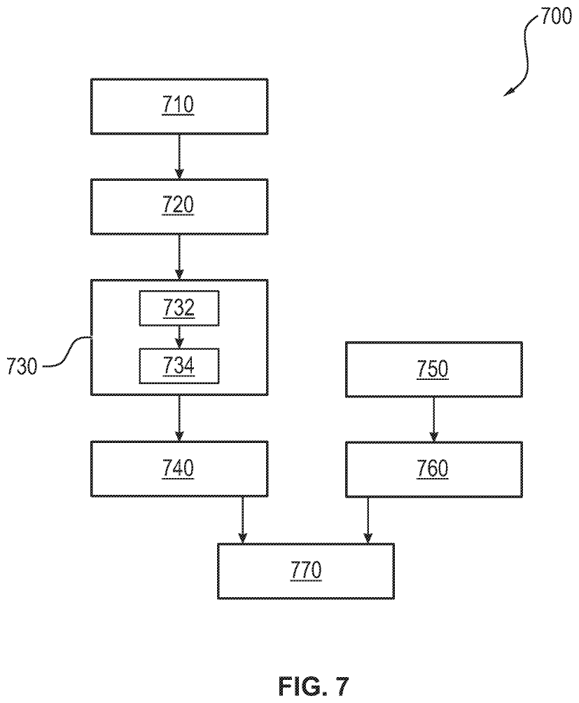

FIG. 7 shows a flow chart of a first exemplary embodiment of a process 700 according to the present invention.

The process 700 according to the present invention for displaying an output of an electromedical device comprises a sequence of the following described steps:

A first step 710 comprises the provision of a user interface, via which a user can provide a user input.

Another step 720 comprises the reception of device data that pertain to the electromedical device.

A next step 730 comprises in a first partial step 732 the assignment of the received device data to a respective visualization type and the dynamic determination of a respective display type of the received device data based on the visualization types to be displayed currently in a second partial step 734.

A next step 740 comprises the generation and outputting of a first display signal based on the device data received and on the display type determined in each case.

A step 750 independent of the previous steps comprises the reading out of data set data.

A next step 760 comprises the generation and outputting of a second display signal based on the data set data. The second display signal indicates a display of a logon screen to be filled out in case an authorization state assigned to these data types is not currently present for currently read-out data set data from a predefined subset of data types.

A final step 770 comprises the reception of the first display signal and of the second display signal and the displaying of a combined visual output based on the two display signals.

Step 710 is typically carried out once during the manufacture of the corresponding device.

Steps 720 through 740 are preferably carried out following one another, wherein the reception of device data corresponding to step 720 triggers the other steps. Steps 750 and 760 following one another are preferably carried out independently of steps 720 through 740 due to the reception of the data set data.

Steps 720 through 740 and 770 are preferably carried out at regular time intervals, which are selected such that the combined visual output is carried out almost in real time with the reception of the data.

In case no data set data were read out, a second display signal, which indicates the information that the provision of data set data has not been carried out, is nevertheless triggered. In an exemplary embodiment, not shown, a second display signal is not outputted in case no data set data were received. Correspondingly, only the first display signal is taken into consideration in the display of the combined visual output.

While specific embodiments of the invention have been shown and described in detail to illustrate the application of the principles of the invention, it will be understood that the invention may be embodied otherwise without departing from such principles.

### LIST OF REFERENCE NUMBERS

100, 200, 300 Display system
105, 105′, 105″, 105′″ Electromedical device
107 Device data
108 Device signal
110 Display unit
112 First display signal
114 Second display signal
116 Combined visual output
118 Display
120 Input unit
122 User interface
124 User input
126 Authorization signal
130, 330 Display signal unit
132 Communication interface
134 First processing unit
135 Second processing unit
136 Third processing unit
140 Data processing unit
142 Data set data
144 Logon screen
250 Display signal monitoring unit
251 Comparison step
252 Output signal
253 Checking step
254 Warning signal
260, 360 Housing
350 Communication network
370 Separate housing
375 Bidirectional wireless connection
480, 680 Administrative output
482 Administrative output area
485 Medical output
490, 490′, 490″ Output area
592 Display of an incomplete output
694 Shifted window
700 Process
710, 720, 730, 740, Process steps
750, 760, 770
732, 734 Partial steps of process step 730

What is claimed is:

1. A display system for displaying an output of at least one electromedical device, the display system comprising:
a display unit configured to receive a first display signal and at least one second display signal and to display a combined visual output based on the two display signals;
an input unit comprising a user interface, via which a user provides a user input;
a display signal unit with a communication interface for communication with the electromedical device, the display signal unit being configured to receive electromedical device data via the communication interface, to assign the electromedical device data to a visualization type, and to determine a respective display type of the received device data dynamically as a function of the visualization types to be displayed currently and to generate the first display signal based on the device data and the respectively determined display type and to output the first display signal to the display unit; and
a data processing unit configured to read out data set data and to output and generate the second display signal from the data set data and if an authorization state of the display system assigned to data types is not currently present for currently read out data set data from a predefined subset of data types, to output the second display signal to provide a logon screen to be filled out by a user of the display system, via the input unit.

2. A display system in accordance with claim 1, further comprising a display signal monitoring unit signal connected to the display unit and to the display signal unit, wherein:
the display signal monitoring unit is configured to receive the first display signal from the display signal unit and to receive an output signal from the display unit;
the output signal contains information about the combined visual output carried out; and
the display signal monitoring unit is configured to compare the first display signal and the output signal with one another based on the respectively determined display type and to output a warning signal in case of a lack of agreement of the respectively determined display type.

3. A display system in accordance with claim 2, wherein the display signal monitoring unit is further configured to check whether or not the output signal has device data or data set data of a visualization type, which may be outputted exclusively in the presence of a defined authorization state and to output a corresponding warning signal if this authorization state is not currently present.

4. A display system in accordance with claim 1, wherein:
the data processing unit is further configured to receive an authorization signal as a function of the user input and to determine a currently present authorization state of the display system as a function of the authorization signal; and
the corresponding data set data are outputted via the second display signal for a predefined subset of data types only if an authorization state assigned to these data types is currently present.

5. A display system in accordance with claim 1, wherein the predefined subset of data types comprises personal data of a person being treated with the electromedical device.

6. A display system in accordance with claim 1, wherein the display signal unit is further configured to generate the first display signal such that a corresponding reference is provided via the combined visual output in case not all device data received from the electromedical device can be visually outputted by the display unit because of the dimensions of the combined visual output.

7. A display system in accordance with claim 1, wherein the display signal unit is further configured to determine a display size and/or a display position of received device data of a visualization type as a function of a relevance level assigned to this visualization type.

8. A display system in accordance with claim 1, wherein:
the communication interface is configured to receive device data from a plurality of electromedical devices;
the visualization type assigned to the device data by the display signal unit comprises device information that indicates the electromedical device corresponding to the device data.

9. A process for displaying an output of at least one electromedical device, the process comprising the steps of:
providing a user interface, via which a user can provide a user input;
receiving device data that pertain to the at least one electromedical device;
assigning the received device data to a respective visualization type and dynamically determining a corresponding display type of the received device data based on the visualization types to be displayed currently;
generating and outputting a first display signal based on the received device data and based on the respectively determined display type;
reading out of data set data;
generating and outputting a second display signal based on the data set data read, wherein the second display signal indicates a display of a logon screen to be filled out in case an authorization state assigned to the data types is not currently present for currently read-out data set data from a predefined subset of data types; and
receiving the first display signal and of the second display signal and displaying a combined visual output based on the two display signals.

10. A process in accordance with claim 9, further comprising the steps of:
sending a combined visual output signal, which contains information about the combined visual output;
receiving the combined visual output signal and the first display signal;
comparing the received combined visual output signal and the first display signal with one another; and
outputting a warning signal if there is a lack of agreement between the combined visual output, represented by the combined visual output signal and a visual output represented by the first display signal.

11. A process in accordance with claim 10, further comprising the steps of:
checking whether or not the combined visual output signal has device data or data set data of a visualization type, which may be outputted exclusively in the presence of a defined authorization state; and
outputting a corresponding warning signal in case this authorization state is not currently present.

12. A process in accordance with claim 9, further comprising the steps of:
receiving an authorization signal as a function of the user input; and
determining a currently present authorization state based on the authorization signal, wherein the corresponding data set data are outputted via the second display signal for a predefined subset of data types only if an authorization state assigned to these data types is currently present.

13. A process in accordance with claim 9, wherein the process is configured to display an output of a plurality of electromedical devices, and further comprising the step of receiving device data that pertain to at least one additional electromedical device, wherein the visualization type assigned to the device data comprises device information that indicates the electromedical device corresponding to the device data.

14. A process according to claim 9, further comprising:
providing a program with program code for carrying out the steps of receiving device data, assigning the received device data to the respective visualization type and dynamically determining the corresponding display type of the received device data based on the visualization types to be displayed currently, generating and outputting the first display signal, reading out of data set data, generating and outputting the second display signal and receiving the first display signal and of the second display signal and providing a display signal for displaying a combined visual output based on the two display signals; and,
running the program code on a computer, on a processor or on a programmable hardware component.
